(19) United States
(12) Patent Application Publication
(10) Pub. No.: US 2015/0100033 A1
(43) Pub. Date: Apr. 9, 2015

(54) SEMI-RIGID DEVICE FOR SEALING AND ALLOWING CONTINUOUS DRAINAGE OF COLOSTOMY BAGS

(71) Applicants: Kristopher Mark Weide, Eldorado Hills, CA (US); Michael Henry Calouette, Gladstone, MI (US)

(72) Inventors: Kristopher Mark Weide, Eldorado Hills, CA (US); Michael Henry Calouette, Gladstone, MI (US)

(21) Appl. No.: 14/045,108

(22) Filed: Oct. 3, 2013

(51) Int. Cl. A61F 5/445

(52) U.S. Cl. CPC A61F 5/445
USPC 604/335

(57) ABSTRACT

An ostomy drainage device that is semi-rigid. The device has a semi-rigid post that has a defined fluid channel in its top surface. The device provides a leak proof seal while the semi-rigid post with the fluid channel allows for the flow of fluid and prevents the possibility of the bag pinching closed or even reducing fluid flow. Other devices do not teach the ability to prevent the stoppage of flow or even prevent limiting of the flow.

### BACKGROUND OF THE INVENTION

The applicants are aware of the following prior art. In U.S. Pat. No. 3,841,332 entitled, “Enterostomy Drainage Appliance”, issued to Dennis L. Treacle on Oct. 15, 1974 teaches a drainage bag with a rib assembly having a generally oblong, open ended, U-shaped configuration mounted on the front wall of the bag within the fluid chamber to prevent sealing of the stoma-receiving opening and bottom drain opening. A plurality of slits are formed in the rib assembly giving it the flexibility to bend with the movements of the body. It has been discovered that the rib portion can still be closed off or crush closed by body movement because the ribbed portion is not rigid enough.

Another example of a prior art device can be found in U.S. Pat. No. 2,496,175, entitled, “Stoma Receiver” issued to M. Perry on Jun. 21, 1946 which teaches a device for receiving a stoma. This prior art device is a prime example of a receiver and a bag that will close off when a patient bends over. The bag is flexible and will completely close off when it is pinched by body movement. The present invention allows the user to make body movements that would clearly close off this prior at device. Again, the semi rigid construction allows the user to bend, but because there is a defined fluid path in the surface of the present device the fluid is never stopped.

Another example of prior art would include U.S. Pat. No. 4,634,437, entitled “Anti-kink Device for a Flexible Urine Collector”, issued to Peter T. Lowthian on Jan. 6, 1987. This patent teaches a device for preventing or hindering obstruction of an inlet valve of a urine collection bag. The central limb of the device is curved in cross section and seats the valve so that even when the bag or valve is crushed, twisted or shortened, the valve remains open. This is simply not true. The central limb does not extend far enough into the bag to eliminate complete closing off of the bag while in use. It has been discovered that the length of the present invention does in fact prevent complete closure. The flexible, semi rigid device extends far enough within the bag to prevent complete closure and that the molded defined fluid path prevents closure at all time, not just some of the time.

The present invention is more rigid yet still flexible. The present invention is rigid enough to resist closure because it has been discovered that the fluid path is molded into the surface and is incapable of closure by body movement. Yet the device is still flexible enough to allow complete body movement. The rigid construction and defined fluid path give the present invention greater utility over Treacle.

The present invention teaches a flexible, semi-rigid device with a molded fluid path in its surface that that extends well into the ostomy bag to prevent any of the short comings of the prior art devices.

### THE INVENTION

The present invention is an ostomy drainage device comprised of a flange. The flange has an opening therethrough. The flange has a top surface and a drainage post. The drainage post has a central drainage channel from the opening to a distal end of the drainage post. The channel has stabilizer bars on each edge of the channel. The ostomy drainage device is formed of a semi-rigid material having a Shore A Durometer of 40 to 80.

### BRIEF DESCRIPTION OF THE DRAWINGS

FIG. 1 shows the ostomy drainage device.

FIG. 2 shows the ostomy drainage device from the side.

FIG. 3 shows the ostomy drainage device from the top.

FIG. 4 shows the ostomy drainage device from the side.

FIG. 5 shows the ostomy drainage device from the end.

FIG. 6 shows a second embodiment of the ostomy drainage device with the double flange.

FIG. 7 shows the second embodiment of the ostomy drainage device with the double flange from the side.

FIG. 8 shows the second embodiment of the ostomy drainage device with the double flange from the top.

FIG. 9 shows the second embodiment of the ostomy drainage device with the double flange from the side.

FIG. 10 shows the second embodiment of the ostomy drainage device with the double flange from the end.

FIG. 11 shows the third embodiment of the ostomy drainage device with the end tab.

FIG. 12 shows the third embodiment of the ostomy drainage device with the end tab from the side.

FIG. 13 shows the third embodiment of the ostomy drainage device with the end tab from the top.

FIG. 14 shows the third embodiment of the ostomy drainage device with the end tab from the side.

FIG. 15 shows the third embodiment of the ostomy drainage device with the end tab from the end.

FIG. 16 shows the fourth embodiment of the ostomy drainage device without a flange.

FIG. 17 shows the fourth embodiment of the ostomy drainage device without a flange tram the side.

FIG. 18 shows the fourth embodiment of the ostomy drainage device without a flange from the top.

FIG. 19 shows the fourth embodiment of the ostomy drainage device without a flange from the side.

FIG. 20 shows the fourth embodiment of the ostomy drainage device without a flange from the end.

### DETAILED DESCRIPTION OF THE DRAWINGS

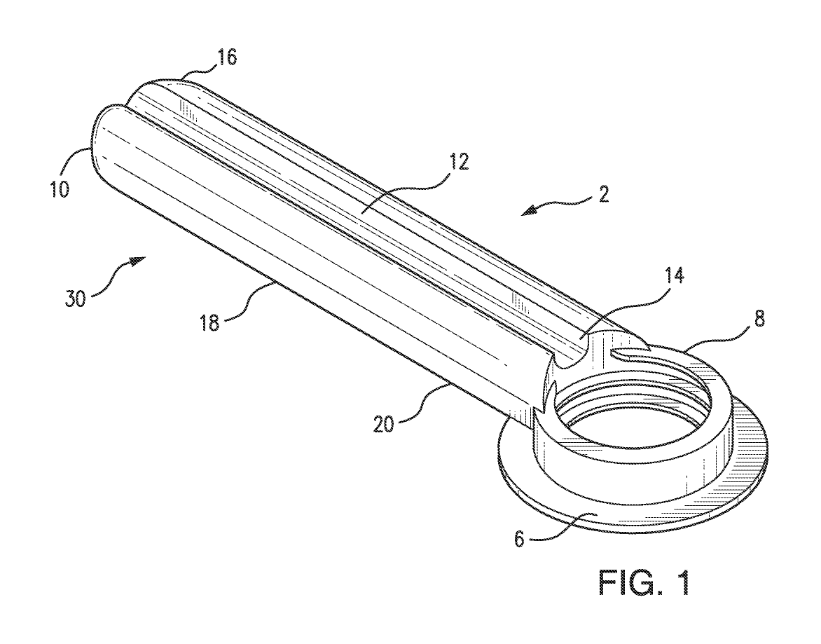

FIG. 1 shows the ostomy drainage device 2. This embodiment allows for easier installation for users with limited physical dexterity while still promoting a sealing surface between the stomata and the point of entry in the bag. The single flange relies on the stomata adhesive around the hole provided in the bag to promote a sealing surface for potential leaks.

The present device 2 seals around a stoma of a patient and extends downward within the colostomy bag of a patient. The semi rigid construction allows the patient to move freely without pinching or stopping the flaw of liquid from within the bag at all times. It has been discovered that the defined pathway 12 or channel on the front surface 18 prevents stoppage of fluid flow even when the patient is bent over. The bag will start to collapse as the patient's body puts pressure on the bag. Again, this defined pathway 12 is unobstructed. The ostomy drainage device 2 has a flange 6 that seals around the stoma. The flange 6 is near the near end 8. One edge of the flange 6 has the near end 14 of the defined path 12. The distal end 16 of the pathway 12 extends down within the bag. Typically the colostomy bags drain from the bottom. It has been discovered that the seal around the stoma by the flange 6 and with the semi rigid device 2 extending toward the bottom of the bag that there is always present a pathway 12 for fluid flow creating a greater utility over any prior art device.

The portion of the device 2 that is below the opening therethrough 22 and the flange 6 is considered the drainage post 30.

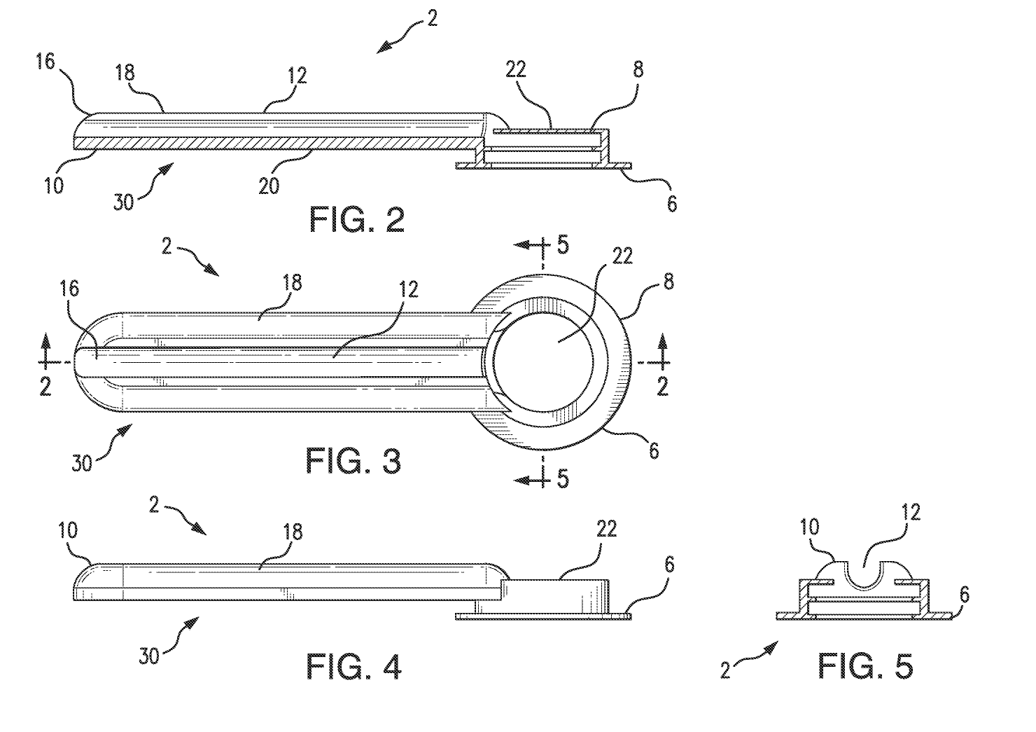

FIG. 2 shows the ostomy drainage device 2 from the side. It is clear that the opening therethrough 22 encircles the stoma of the patient and the flange 6 makes a seal against the inside of the bag and the patient's skin eliminating any leakage. Also clear is the front surface 18 and the back surface 20. This figure shows the defined path 12 in the front surface 18. It is clear to see that the pathway 12 will remain open even when a bag places pressure against the device because the pathway 12 remains below the front surface 18 keeping the pathway 12 always open. The ability of the bag to prevent flow out is eliminated. The pathway 12 is continuously unobstructed.

FIG. 3 shows the ostomy drainage device 2 from the top. Here it is clear the relationship between the near end 14 of the pathway 12 and the opening therethrough 22. The fluid leaves the patient's body through the stoma and past the flange 6 to the pathway 12 toward the drain in the bottom of the bag. The fluid will flow down the pathway 12 toward the distal end 16 of the pathway 12. After the fluid flows past the distal end 16 of the pathway 12 at the distal end 10 of the device 2 it continues into the bag and heads out toward the drain unobstructed.

FIG. 4 shows the ostomy drainage device 2 from the side. This figure shows the flange 6. The device 2 is placed within a colostomy bag, then over the patient's stoma providing unobstructed drainage along the front surface 18 toward the distal end 10.

FIG. 5 shows the ostomy drainage device from the end. This figure shows the distal end 10 of the device 2. This is the point where fluid will exit the device 2 through the pathway 12 and flow into the bag. The flange 6 is also visible. It is clear that the seal will attach around the patients stoma inside the bag and make a seal against the patient's skin preventing any leakage.

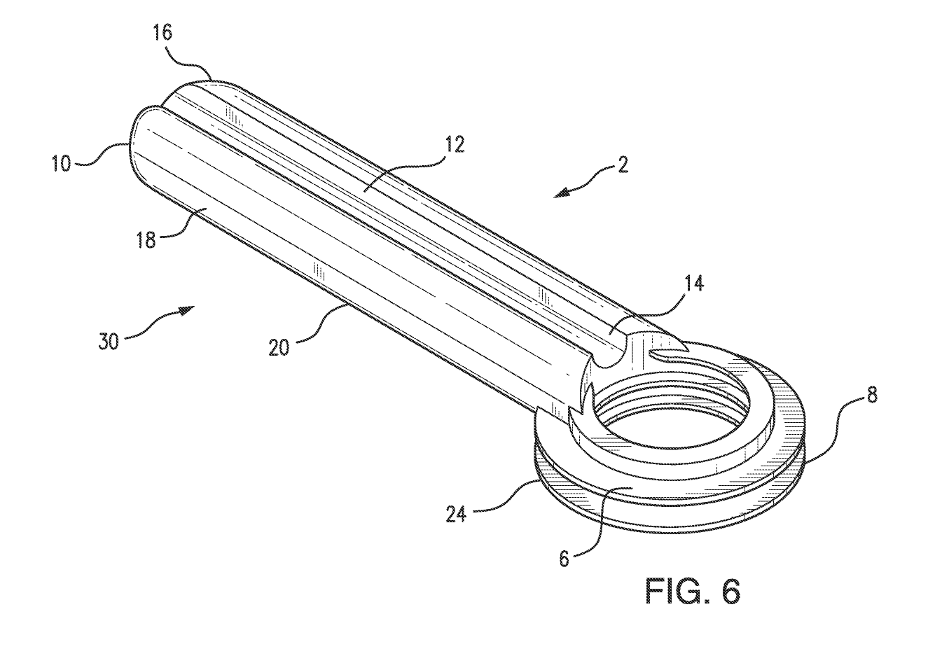

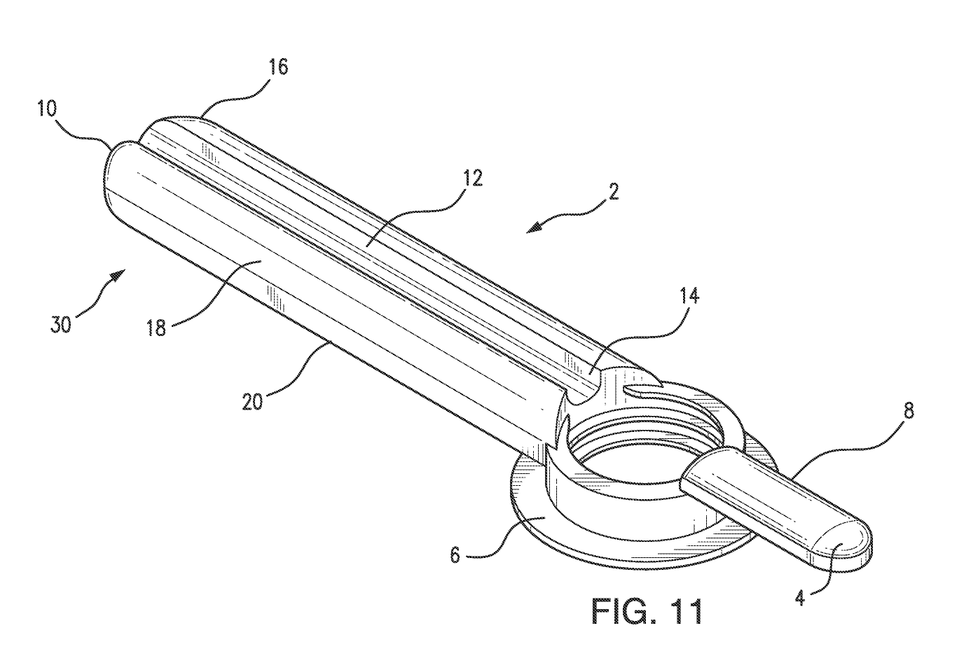

FIG. 6 shows a second embodiment of the ostomy drainage device 2. This embodiment is designed for colostomy bags that will not accommodate the stabilizer tab or end tab 4 (FIG. 11). It has been discovered that the defined pathway 12 on the front surface 18 prevents stoppage of fluid flow even when the patient is bent over. The bag will start to collapse as the patient's body puts pressure on the bag. Again, this defined pathway 12 remains unobstructed. This embodiment allows the stomata to be locked into place keeping the stoma and the opening inside diameter concentric while in use. Once the double flange 6 embodiment is in place, the inside diameter of the point of entry remains at a constant size without deformation providing a sealing surface even if the stoma shrinks or increases in size. The double flange 6 of this embodiment is a first flange 6 with a second flange 6 surmounted on it and is generally referred to as the flange 6.

The ostomy drainage device 2 has a flange 6 that seals around the stoma. The flange 6 is near the near end 8. One edge of the flange 6 has the near end 14 of the defined path 12 terminus. The distal end 16 of the pathway 12 extends down within the bag. Typically the colostomy bags drain from the bottom. It has been discovered that the seal around the stoma by the flange 6 and with the semi rigid device 2 extending toward the bottom of the bag that there is always present a pathway 12 for fluid flow creating a greater utility over any prior art device.

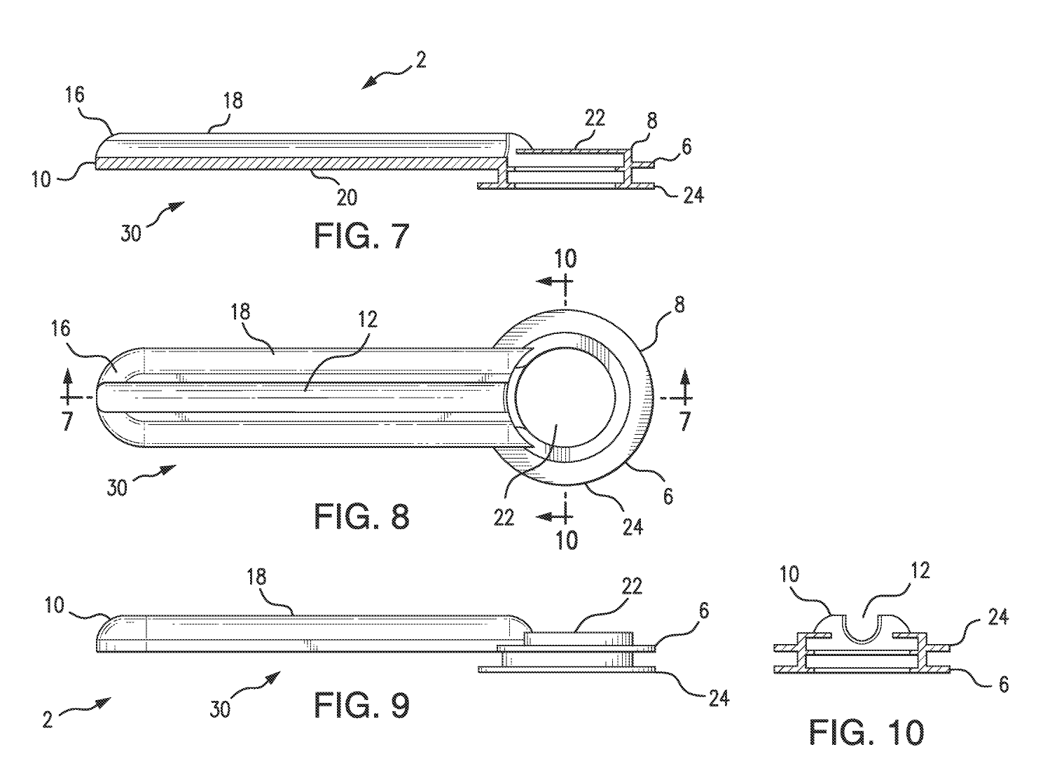

FIG. 7 shows the second embodiment of the ostomy drainage device 2 from the side. The reason this embodiment does not have the end tab 4 is to accommodate different colostomy bags. Some bags have a lesser distance to the top of the colostomy bag from the opening that accommodates the stoma. With this in mind, this embodiment allows for the same seal around a stoma of a patient and extends downward within the colostomy bag of a patient. The semi rigid construction allows the patient to move freely without pinching or stopping the flow of liquid from within the bag at all times. It has been discovered that the defined pathway 12 on the front surface 18 prevents stoppage of fluid flow even when the patient is bent over. The bag will start to collapse as the patient's body puts pressure on the bag. Again, this defined pathway 12 is unobstructed. The ostomy drainage device 2 has a flange 6 that seals around the stoma. The flange 6 is near the near end. One edge of the flange 6 has the near end 14 of the defined path. The distal end 16 of the pathway 12 extends down within the bag. Typically the colostomy bags drain from the bottom. It has been discovered that the seal around the stoma by the flange 6 and with the semi rigid device 2 extending toward the bottom of the bag that there is always present a pathway 12 for fluid flow creating a greater utility over any prior art device. Again, the main difference here is to accommodate a bag that has less distance between the opening for the stoma and the top of the bag.

FIG. 8 shows the second embodiment of the ostomy drainage device 2 from the top. The top view emphasizes the double flange 6 that allows for the device 2 to essentially lock into place. It will also fit a stoma that is longer than others. It should be understood that all stomas are not the same in diameter and in length. Another utility of this device is to help accommodate differing stomas with one device.

FIG. 9 shows the second embodiment of the ostomy drainage device 2 from the side. Again, this embodiment eliminates the end tab 4 to also accommodate the lack of space between the stoma opening and the top end of the bag.

FIG. 10 shows the second embodiment of the ostomy drainage device 2 from the end. This figure shows the distal end 10 of the device 2. This is the point where fluid will exit the device through the pathway 12 and flow into the bag. The flange 6 is also visible. It is clear that the seal will attach around the patients stoma and make a seal against the inside of the bag and the patient's skin preventing any leakage.

FIG. 11 shows the third embodiment of the ostomy drainage device 2. The present device 2 seals around a stoma of a patient and extends downward within the colostomy bag of a patient. The semi rigid construction allows the patient to move freely without pinching or stopping the flow of liquid from within the bag at all times. It has been discovered that the defined pathway 12 on the front surface 18 prevents stoppage of fluid flow even when the patient is bent over. The bag will start to collapse as the patient's body puts pressure on the bag. Again, this defined pathway 12 is unobstructed. The ostomy drainage device has a flange 6 that seals around the stoma. The flange 6 is near the near end. One edge of the flange 6 has the near end 14 of the defined path. The distal end 16 of the pathway 12 extends down within the bag. Typically the colostomy bags drain from the bottom. It has been discovered that the seal around the stoma by the flange 6 and with the semi rigid device 2 extending toward the bottom of the bag that there is always present a pathway 12 for fluid flow creating a greater utility over any prior art device.

This embodiment promotes flow control off the top side of the stoma. Depending on the placement of the stoma on the patient's abdomen or how active the user is, fluid can build up in the upper portion of the bag causing leakage around the stoma. The upper extrusion or stabilizer tab 4 of the stomata acts like the lower extrusion by giving the fluid a path to drain into the larger capacity section of the bag.

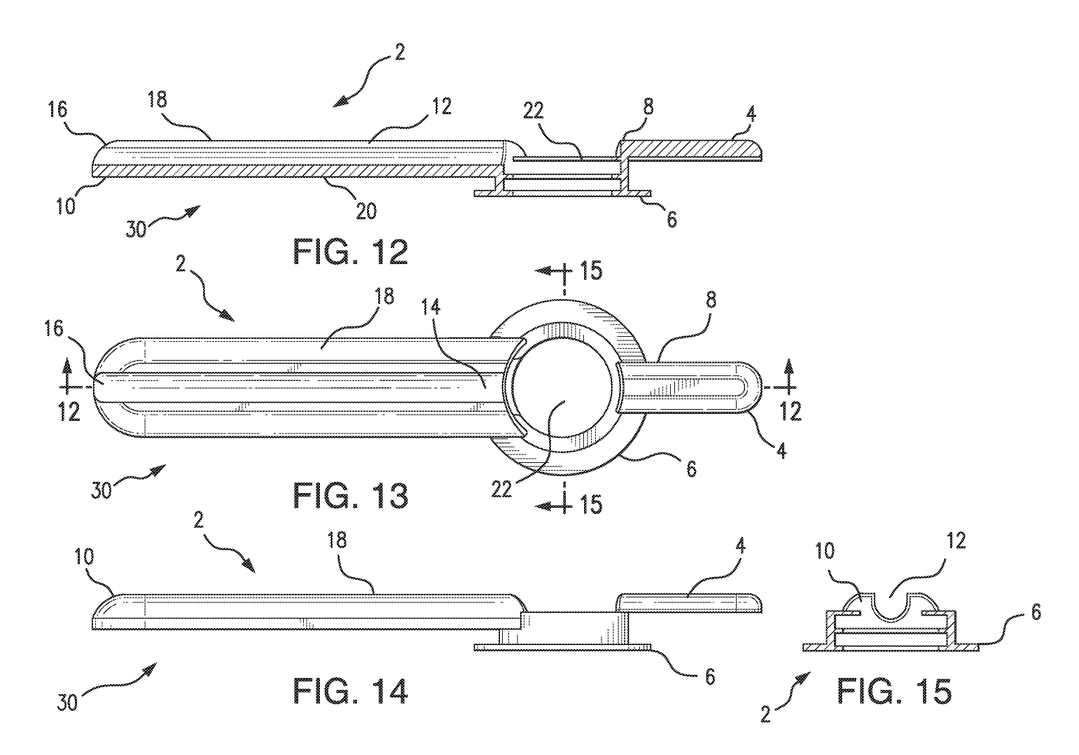

FIG. 12 shows the third embodiment of the ostomy drainage device 2 from the side. It is clear that the opening therethrouqh 22 encircles the stoma of the patient and the flange 6 makes a seal against the inside of the bag and the patient's skin eliminating any leakage. The end tab 4 extends beyond the opening therethrough 22 to stabilize the device 2 and allow for increase capacity utilization in the top portion of the bag. Also clear is the front surface 18 and the back surface 20. This figure shows the defined path. 12 in the front surface 18. It is clear to see that the pathway 12 will remain open even when a bag places pressure against the device because the pathway 12 remains below the front surface 18 keeping the pathway 12 always open.

FIG. 13 shows the third embodiment of the ostomy drainage device 2 from the top. Here it is clear the relationship between the near end 14 of the pathway 12 and the opening therethrough 22. As fluid leaves the patient's body through the stoma, past the flange 6 it can takes the pathway 12 toward the drain in the bottom of the bag. The fluid will flow down the pathway 12 toward the distal end 16 of the pathway 12. After the fluid flows past the distal end 16 of the pathway 12 at the distal end 10 of the device 2 it continues into the bag and heads out toward the drain unobstructed.

FIG. 14 shows the third embodiment of the ostomy drainage device 2 from the side. This figure shows the stabilizer tab 4 and the flange 6. The device 2 is placed within a colostomy bag, then over the patient's stoma providing unobstructed drainage along the front surface 18 toward the distal end 10.

FIG. 15 shows the third embodiment of the ostomy drainage device 2 from the end. This figure shows the distal end 10 of the device 2. This is the point where fluid will exit the device 2 through the pathway 12 and flow into the bag. The flange 6 is also visible.

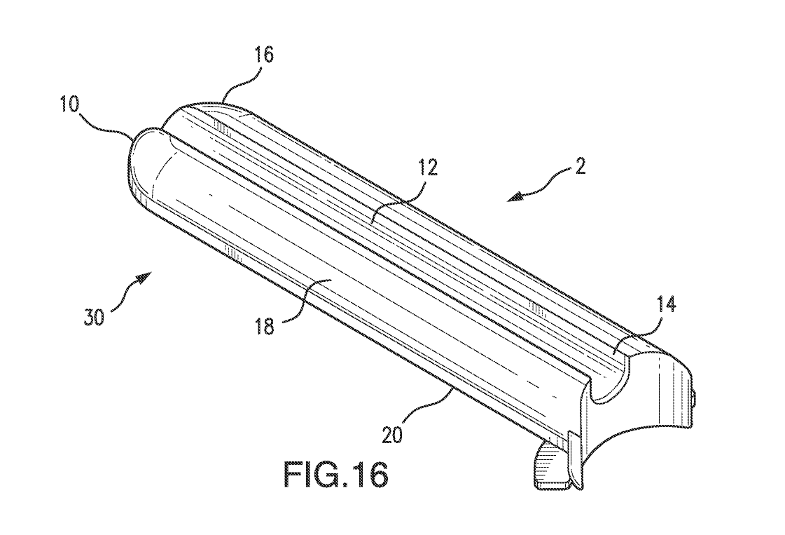

FIG. 16 shows the fourth embodiment of the ostomy drainage device 2. This embodiment addresses concerns for user's that are less concerned about leakage and more concerned with maximum capacity. They are less concerned with leakage but want the assurance that the bag itself will work to 100% of capacity. This embodiment also addresses the concern that the stomas have varying sizes. This embodiment does not have to be sized to the user's stoma and is the easiest of all the embodiments to install.

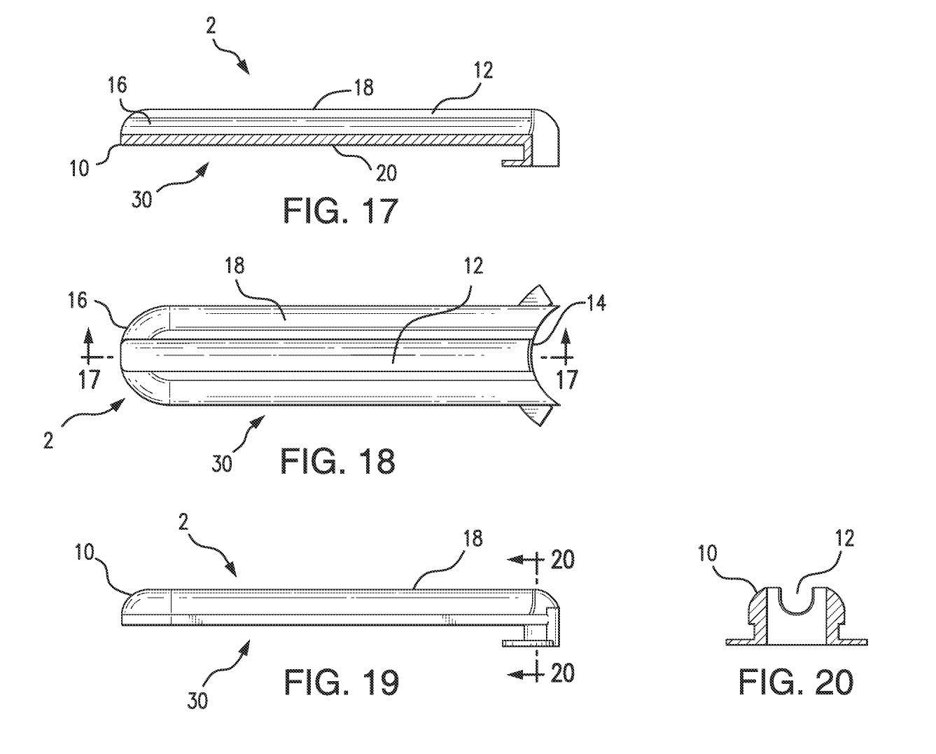

FIG. 17 shows the fourth embodiment of the ostomy drainage device 2 from the side. This embodiment has no flange 6 or is considered semi flanged. However, it does provide the pathway 12 for fluid flow along the top surface 18 toward the distal end 16 of the distal end 10 of the device 2. Essentially the device operates the same as the other embodiments without the flange portion 6.

FIG. 18 shows the fourth embodiment of the ostomy drainage device 2 from the top. Again, it is very clear that the pathway 12 in the top surface 18 cannot become obstructed by the bag regardless of the patient's movements or positioning.

FIG. 19 shows the fourth embodiment of the ostomy drainage device 2 from the side. This embodiment allows the user to use the device 2 in smaller bags or bags that do not have head room within the bag.

FIG. 20 shows the fourth embodiment of the ostomy drainage device 2 from the end. The end view show the pathway 12 within the top surface 18 and how it will remain open under any circumstance during use of the device within a colostomy bag. The present ostomy drainage device is formed of a semi-rigid material having a Shore A Durometer of 40+/−5 to 85+/−5.

The device is constructed flexible enough to allow complete body movement. The rigid construction and defined fluid path give the present invention greater utility over any prior art.

Again, the semi rigid construction allows the user to bend, but because there is a defined fluid path 12 in the surface of the present device the fluid is never stopped. It has been discovered that the length of the present invention does in fact prevent complete closure. The flexible, semi rigid device extends far enough within the bag to prevent complete closure and that the molded defined fluid path 12 prevents closure at all time, not just some of the time. The present invention teaches a flexible, semi-rigid device with a molded fluid path 12 in its surface that that extends well into the ostomy bag to prevent any of the short comings of the prior art devices.

What is claimed is:

1. An ostomy drainage device comprising:
a. a flange, said flange having an opening therethrough, said flange having a top surface;
b. a drainage post, said drainage post having a central drainage channel from said opening to a distal end of said drainage post;
c. said channel having stabilizer bars on each edge of said channel;
d. said ostomy drainage device being formed of a semi-rigid material having a Shore A Durometer of 40+/−5 to 85+/−5.

2. The ostomy drainage device as claimed in claim 1 wherein, in addition, there is a stabilizer bar fixedly attached to said top surface, opposite said channel.

3. The ostomy drainage device as claimed in claim 1 wherein said flange is a double flange consisting of a first flange surmounting a second flange.

4. The ostomy drainage device as claimed in claim 1 wherein, said flange is a semi-flange, wherein said semi-flange is located at said drainage post.
